(19) United States
(12) Patent Application Publication
(10) Pub. No.: US 2015/0366575 A1
(43) Pub. Date: Dec. 24, 2015

(54) LIQUID EJECTION DEVICE

(71) Applicant: SEIKO EPSON CORPORATION, Tokyo (JP)

(72) Inventors: Motohiko SATO, Sendai-shi (JP); Atsuhiro NAKAGAWA, Sendai-shi (JP); Atsushi NAKAYASHIKI, Sendai-shi (JP); Tominaga Teiji, Sendai-shi (JP); Hideki KOJIMA, Matsumoto-shi (JP)

(21) Appl. No.: 14/745,304

(22) Filed: Jun. 19, 2015

(30) Foreign Application Priority Data
Jun. 20, 2014 (JP) 2014-127001

(51) Int. Cl. A61B 17/3203

(52) U.S. Cl. CPC A61B 17/3203

(57) ABSTRACT

A liquid ejection device includes: a liquid ejection opening configured to eject a first liquid; a liquid suction opening arranged in the vicinity of the liquid ejection opening; and a suction unit configured to suck liquid from the liquid suction opening via a suction tube, wherein the liquid ejection device includes a second liquid supply unit, and a second liquid can be supplied into the suction tube.

This application claims the benefit of Japanese Patent Application No. 2014-127001, filed on Jun. 20, 2014. The content of the aforementioned application is incorporated herein by reference in its entirety.

### BACKGROUND

1. Technical Field

The present invention relates to a liquid ejection device and a surgical facility.

2. Related Art

A medical apparatus configured to treat an affected area by applying ejected fluid to the affected area is used. For example, a liquid ejection device is disclosed in JP-A-1-313047. The liquid ejection device is used as a surgical knife for hospital use, for example, and is capable of excising or breaking up a living body by continuously ejecting a liquid such as a normal saline solution toward the living body. In addition, a liquid ejection device provided with a suction port and a suction route connected to a suction apparatus in order to suck liquid such as a normal saline solution ejected toward the living body (operated portion) or liquid in the periphery of the operated portion is considered. In this configuration, the liquid is not accumulated in the operated portion, and visibility of the operated portion for an operator may be secured (JP-A-1-313047 and JP-A-6-90957). Also, a liquid ejection device which is configured to eject the liquid such as the normal saline solution toward the living body in a pulsed manner so that excising with a lower flow rate than the rate required for ejecting the liquid continuously is enabled is known (JP-A-2011-177407).

In JP-A-1-313047 and JP-A-6-90957, a living tissue excised by ejecting a normal saline solution continuously toward the living body, and the ejected liquid are sucked by using the sucking apparatus. In JP-A-2011-177407, since the normal saline solution or the like is ejected toward the living body in a pulsed manner, the amount of liquid ejected toward the operated portion is relatively smaller than that in the case where the normal saline solution or the like is continuously ejected in JP-A-1-313047 and JP-A-6-90957. Consequently, with the liquid ejection device configured to eject the normal saline solution or the like toward the living body in a pulsed manner, the amount of liquid sucked by using the sucking apparatus is relatively smaller than that of the liquid ejection device configured to eject the normal saline solution or the like continuously. Therefore, the liquid ejection device configured to eject the normal saline solution or the like toward the living body in a pulsed manner has a tendency that the living tissue or the like excised and sucked from the living body accumulates in the suction route and hence the suction route is clogged in comparison with the liquid ejection device configured to eject the normal saline solution or the like continuously.

Therefore, a liquid ejection device configured to eject the normal saline solution or the like toward the living body in a pulsed manner provided with a suction route configured to cause less clogging of excised living tissue or ejected liquid is desired.

### SUMMARY

An advantage of some aspects of the invention is to solve at least a part of the problems described above, and the invention can be implemented as the following forms or application examples.

### APPLICATION EXAMPLE 1

This application example is directed to a liquid ejection device including: a liquid ejection opening configured to eject a first liquid; a liquid suction opening arranged in the vicinity of the liquid ejection opening; and a suction unit configured to suck liquid from the liquid suction opening via a suction tube, wherein the liquid ejection device includes a second liquid supply unit, and a second liquid can be supplied into the suction tube.

According to this application example, since the second liquid is supplied into the suction tube, a sucked tissue is not likely to be accumulated in the suction tube owing to a water flow caused by suction, so that the suction tube which is not likely to be clogged is achieved.

### APPLICATION EXAMPLE 2

This application example is directed to the liquid ejection device according to the application example described above, which further includes a switch configured to switch ejection and non-ejection of the first liquid, and a controller configured to control to make the second liquid supply unit start a supply of the second liquid when starting ejection of the first liquid, and to stop the supply of the second liquid from the second liquid supply unit when stopping ejection of the first liquid.

According to this application example, since the second liquid is not supplied wastefully when a pulse ejection is not performed, the liquid ejection device which does not waste the second liquid is provided.

### APPLICATION EXAMPLE 3

This application example is directed to the liquid ejection device according to the application example described above, wherein the first liquid is identical with the second liquid.

According to this application example, since the first liquid and the second liquid do not have to be provided separately, the liquid ejection device of a small size is provided.

### APPLICATION EXAMPLE 4

This application example is directed to the liquid ejection device according to the application example described above, wherein the second liquid includes an enzyme which decomposes a protein or a lipid.

According to this application example, since a protein or lipid component of the sucked tissue can be decomposed, the liquid ejection device having a suction route not likely to be clogged is provided.

### APPLICATION EXAMPLE 5

This application example is directed to the liquid ejection device according to the application example described above, wherein a supply flow rate of the second liquid supplied from the second liquid supply unit is 5 milliliters or more per minute.

According to this application example, the sucked tissue is not likely to be accumulated in the suction tube owing to the water flow caused by suction, so that the liquid ejection device in which the sucked tissue is not likely to be accumulated in the suction tube, and the suction route is not likely to be clogged is achieved.

### APPLICATION EXAMPLE 6

This application example is directed to the liquid ejection device according to the application example described above, wherein the liquid ejection device further includes a force supply switch for allowing the second liquid to be supplied from the second liquid supply unit when the liquid is not ejected.

According to this application example, since the interior of the suction tube can be cleaned by the force supply switch, accumulated sucked objects may be flushed, so that the liquid ejection device in which the sucked tissue is not likely to be accumulated in the suction tube, and the suction route is not likely to be clogged is provided.

### BRIEF DESCRIPTION OF THE DRAWINGS

The invention will be described with reference to the accompanying drawings, wherein like numbers reference like elements.

FIGS. 1A and 1B relate to a first embodiment, in which FIG. 1A illustrates a block diagram illustrating a configuration of a liquid ejection device, and FIG. 1B is a partial schematic side view illustrating a structure of a nozzle of the liquid ejection device.

FIG. 2 is a schematic drawing illustrating a flow of a second liquid in a second tube and a suction tube.

FIG. 3 is a schematic cross-sectional view illustrating an internal configuration of a pulsation applying part.

FIG. 4 is a graph illustrating a transition of a capacity of a liquid chamber.

FIG. 5 is an electric control block diagram of the liquid ejection device.

FIG. 6 is a flowchart illustrating a procedure of ejection of the liquid ejection device.

FIG. 7 relates to a second embodiment and shows a block diagram illustrating a configuration of a liquid ejection device.

FIG. 8 relates to a modification of the second embodiment and shows a block diagram illustrating a configuration of a liquid ejection device.

FIG. 9 relates to a third embodiment and shows an electric control block diagram of the liquid ejection device.

### DESCRIPTION OF EXEMPLARY EMBODIMENTS

In embodiments given below, a characteristic liquid ejection device and a characteristic example of a method of excising a living body by using the liquid ejection device will be described with reference to the drawings. Respective members in the drawings have sizes visible in the respective drawings. Therefore, scales in the drawings are differentiating from one member to another.

### First Embodiment

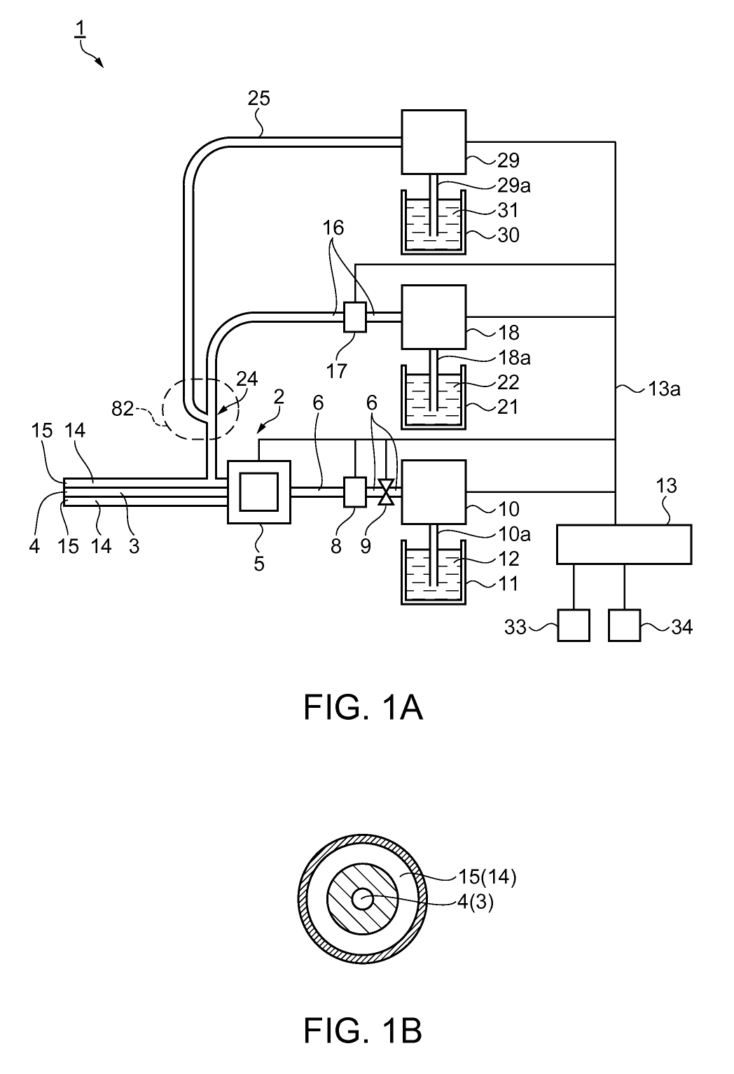

In a first embodiment, a liquid ejection device, which is a surgical facility, will be described with reference to FIGS. 1A to 6. FIG. 1A is a block diagram illustrating a configuration of the liquid ejection device. FIG. 1B is a partial schematic side view illustrating a structure of a nozzle of the liquid ejection device. A liquid ejection device 1 of the first embodiment is a medical apparatus used in a medical organization, and has a function as a surgical knife which incises and excises an affected area by ejecting fluid toward the affected area.

As illustrated in FIG. 1A, the liquid ejection device 1 is provided with a handpiece 2. The handpiece 2 is an instrument that an operator operates in hand when performing an operation. The handpiece 2 is provided with an ejection tube 3, which is a flow channel of a fluid. The ejection tube 3 is provided with a nozzle 4 as a liquid ejection opening configured to eject a fluid at one end thereof. The ejection tube 3 is provided with a pulsation applying part 5 at the other end thereof. A first flowmeter 8, an electromagnetic valve 9, and a first pump 10 are connected to the pulsation applying part 5 in this order via a first tube 6. The pulsation applying part 5 is a portion which makes a fluid passing therethrough a pulse flow.

The first flowmeter 8 measures a flow rate of a fluid flowing in the first tube 6. A hot-wire flowmeter or an impeller flow meter may be used as the first flowmeter 8. Opening and closing of the electromagnetic valve 9 is controlled by an electric signal. A valve configured to be opened and closed by a motor or an electromagnet may be used as the electromagnetic valve 9.

A syringe-type pump or a tube pump may be used as the first pump 10. In the case of the syringe type, an apparatus configured to supply the fluid into the syringe is preferably provided. Accordingly, the liquid ejection device 1 may be driven continuously.

The first pump 10 is provided with a water inlet tube 10a, and one end of the water inlet tube 10a is connected to a first water storage tank 11. A first liquid 12 is contained in the first water storage tank 11. The first liquid 12 may be, for example, a normal saline solution. The normal saline solution, causing no harm to the living body, may be used for a surgical operation.

The liquid ejection device 1 is provided with a control device 13 as a controller, and the control device 13 controls an operation of the liquid ejection device 1. The pulsation applying part 5, the first flowmeter 8, the electromagnetic valve 9, and the first pump 10 are connected to the control device 13 by a cable 13a.

A suction pipe 14 is provided in parallel with the ejection tube 3. A suction port 15 is provided at a distal end of the suction pipe 14 as the liquid suction opening and a suction unit. The suction pipe 14 opens at the suction port 15. The nozzle 4 and the suction port 15 are positioned on substantially the same surface and the suction port 15 is arranged in the vicinity of the nozzle 4. A suction tube 16 is connected to the suction pipe 14. A suction pump 18 is connected to the suction pipe 14 via the suction tube 16 in this order.

In addition, a second tube 25 is connected to the suction tube 16 via a supply port 24, and a second pump 29 is connected to the second tube 25. Although the second pump 29 is not specifically limited, a tube pump, for example, may be used. The second pump 29 is provided with a water inlet tube 29a, and one end of the water inlet tube 29a is connected to a second water storage tank 30. A second liquid 31 is contained in the second water storage tank 30. The second liquid 31 may be, for example, pure water. The second pump 29 is connected to the control device 13 via the cable 13a. A second liquid supply unit is composed of the second pump 29 and the second tube 25.

Pure water or a normal saline solution may be used as the second liquid 31 and, in addition, one of these liquids containing an enzyme which decomposes a protein or a lipid may also be used. In the case where the liquid containing the enzyme which decomposes the protein or the lipid is used, a protein or lipid component such as a living tissue which moves in the suction tube by being sucked is decomposed, so that the living tissue is not likely to be accumulated in the suction tube, and the suction tube is not likely to be clogged.

The suction unit includes a suction flowmeter 17, a water discharge tube 18a, and a drainage tank 21 in addition to the suction pipe 14, the suction tube 16, and the suction pump 18. The suction flowmeter 17 is the similar flowmeter to the first flowmeter 8 and is attached to the suction pump 18 side of the suction tube 16. Although the suction pump 18 is not specifically limited, a tube pump, for example, may be used. The suction pump 18 is provided with the water discharge tube 18a, and the water discharge tube 18a is connected to the drainage tank 21. A drainage fluid 22 drained from the water discharge tube 18a is stored in the drainage tank 21. The suction flowmeter 17 and the suction pump 18 are connected to the control device 13 via the cable 13a.

The control device 13 is provided with a main switch 33 and an ejection switch 34 as a switch. The main switch 33 is configured to activate the liquid ejection device 1. When the main switch 33 is turned ON, an electric power is supplied to the control device 13. The ejection switch 34 is a switch configured to switch ejection and non-ejection of the fluid from the nozzle 4. The ejection switch 34 is configured to be operated by the operator by stepping thereon.

When the operator turns on the main switch 33, the control device 13 is initialized. Then, the operator turns the ejection switch 34 ON. The first pump 10 is activated, and the first pump 10 causes the first liquid 12 to flow to the electromagnetic valve 9. When the control device 13 opens the electromagnetic valve 9, the first liquid 12 at a high pressure proceeds in the first tube 6 as a fluid. Then, the first flowmeter 8 detects the flow rate of the fluid proceeding in the first tube 6 and outputs the flow rate to the control device 13.

The fluid proceeding in the first tube 6 reaches the pulsation applying part 5 via a filter, which is not illustrated. The first liquid 12 reaching the pulsation applying part 5 is subjected to application of pulsation by the pulsation applying part 5. The first liquid 12 passed through the pulsation applying part 5 passes through the ejection tube 3 and is ejected from the nozzle 4. Since pulsation is applied to the first liquid 12 which has passed through the nozzle 4, a pulsed ejection is achieved.

After the operator turns the ejection switch 34 ON, the suction pump 18 is activated in parallel to the activation of the first pump 10. The suction pump 18 sucks excised tissue and a liquid positioned at the suction port 15. The sucked tissue and liquid enter the suction pipe 14 from the suction port 15, pass through the suction tube 16, and are drained into the drainage tank 21.

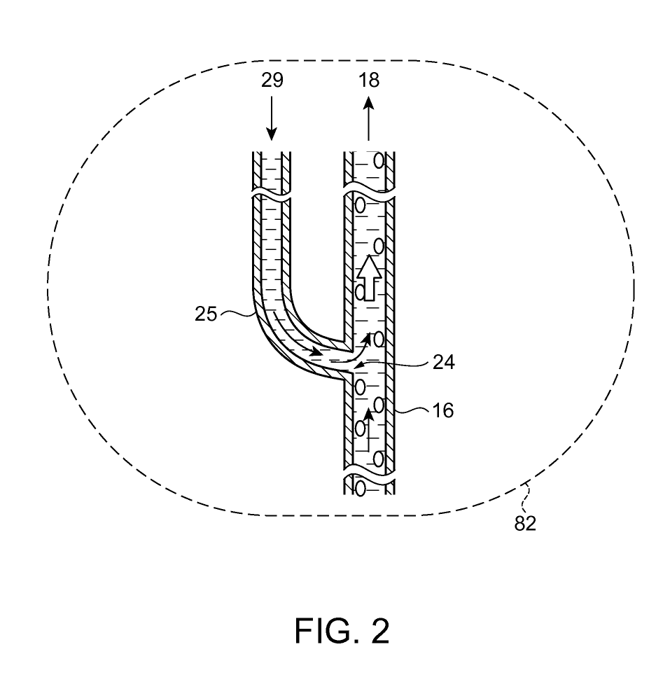

FIG. 2 is a schematic drawing illustrating a peripheral area of the supply port 24 indicated by a circular dot line portion 82 in FIG. 1A in an enlarged scale. Specifically, FIG. 2 is a schematic drawing for explaining a flow of the second liquid 31 in the second tube 25 and the suction tube 16. The second tube 25 is connected to part of the suction tube 16 in a longitudinal direction so as to be capable of communicating with the interior of the suction tube. After the operator turns the ejection switch 34 ON, the second pump 29 is activated in parallel to the activation of the first pump 10. The second pump 29 is activated, and the second pump 29 causes the second liquid 31 to flow to the second tube 25.

Since a suction force is generated in the suction tube 16 when the suction pump 18 is driven, the second liquid 31 passes through the second tube 25, and is supplied to the suction tube 16 via the supply port 24 which is a connecting portion between the second tube 25 and the suction tube 16. The supply port 24 may be arranged within the handpiece 2 or may be arranged outside of the handpiece 2.

As a suction force set in the medical apparatus is on the order of −30 kPa to −70 kPa, typically, on the order of −50 kPa. In contrast, if a supply flowrate of the second liquid 31 supplied from the supply port 24 is set to 5 ml or more per minute, the sucked tissue is not likely to be accumulated owing to the flow of the liquid due to suction, and hence the suction tube is not likely to be clogged.

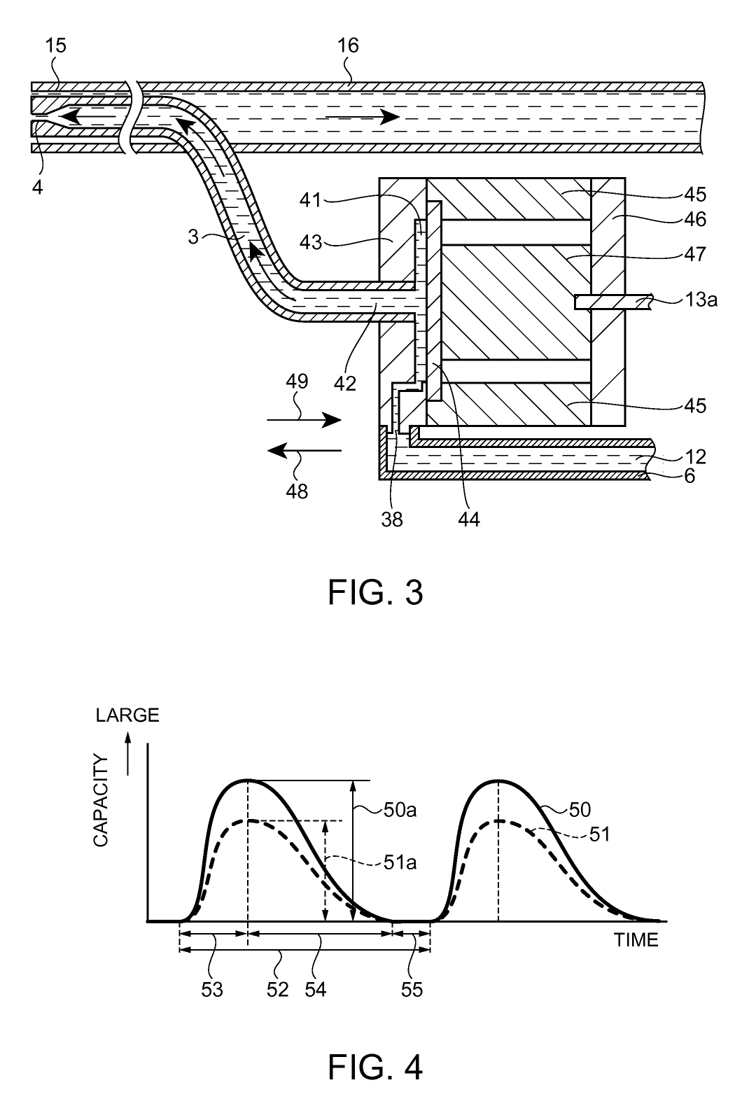

FIG. 3 is a schematic cross-sectional view illustrating an internal configuration of a pulsation applying part. The pulsation applying part 5 is provided with an inlet channel 38, a liquid chamber 41, and an outlet channel 42 in which the first liquid 12 supplied from the first tube 6 passes. The inlet channel 38 and the outlet channel 42 are formed in a first case 43. A diaphragm 44 is provided so as to interpose the liquid chamber 41 between the first case 43 and the diaphragm 44. The first tube 6 is connected to the inlet channel 38, and the ejection tube 3 is connected to the outlet channel 42.

A cylindrical second case 45 is provided on the right side of the first case 43 in the drawing so as to be in contact with the first case 43. The diaphragm 44 is a disc-shaped metallic thin plate, and an outer peripheral portion of the diaphragm 44 is fixedly clamped between the first case 43 and the second case 45. A third case 46 is provided on the right side of the second case 45 in the drawing in contact with the second case 45. A piezoelectric element 47 as a volume varying unit, which is a laminated piezoelectric element, is arranged between the diaphragm 44 and the third case 46. One end of the piezoelectric element 47 is fixed to the diaphragm 44, and the other end thereof is fixed to the third case 46. The piezoelectric element 47 is connected to the control device 13 via the cable 13a.

When a drive voltage is applied from the control device 13, the piezoelectric element 47 changes the capacity of the liquid chamber 41 formed between the diaphragm 44 and the first case 43. When the drive voltage to be applied to the piezoelectric element 47 is increased, the piezoelectric element 47 is expended, and the diaphragm 44 is pushed by the piezoelectric element 47 and is bent toward the liquid chamber 41, which is a first direction 48 in the drawing. When the diaphragm 44 is bent in the first direction 48, the capacity of the liquid chamber 41 is reduced. Then, the fluid in the liquid chamber 41 is pushed out from the liquid chamber 41.

A fluid discharge of the pulsation applying part 5 of the first embodiment is achieved by a difference between an inertance L1 on the inlet channel 38 side (also referred to as a synthetic inertance L1) and an inertance L2 on the outlet channel 42 side (also referred to as a synthetic inertance L2). An inertance L is expressed by L=ρ×h/s where ρ is a fluid density, S is a cross-sectional area of the flow channel, and h is a length of the flow channel. By deforming a motion equation in the flow channel by using the inertance L, a relationship ΔP=L×dQ/dt is derived where ΔP is a pressure difference of the flow channel, and Q is a flow rate of a fluid flowing in the flow channel. In other words, the inertance L indicates a degree of influence on a temporal change of the flow rate, and the larger the inertance L, the less the temporal change of the flow rate, and hence the smaller the inertance L, the larger the temporal change of the flow rate.

In the first embodiment, the length and the cross-sectional area of the inlet flow channel 38 and the length and the cross-sectional area of the outlet channel 42 are set so that the inertance L1 on the inlet channel 38 side becomes larger than the inertance L2 on the outlet channel 42 side. The inlet channel 38 is closer to the first pump 10 than the outlet channel 42, and hence a water pressure in the inlet channel 38 is higher than that in the outlet channel 42. Therefore, major part of the fluid in the liquid chamber 41 passes through the outlet channel 42, and is pushed out from the liquid chamber 41.

In contrast, when the drive voltage to be applied to the piezoelectric element 47 is decreased, the piezoelectric element 47 is contracted, and the diaphragm 44 is pulled by the piezoelectric element 47 and is bent toward the third case 46, which is a second direction 49 in the drawing. The piezoelectric element 47 is contracted and the capacity of the liquid chamber 41 is increased, so that the fluid is supplied from the inlet channel 38 into the liquid chamber 41.

The drive voltage applied to the piezoelectric element 47 repeats ON (maximum voltage) and OFF (0 V) at a high frequency (for example, 300 Hz), so that increase and decrease in capacity of the liquid chamber 41 is repeated, and pulsation is applied to the fluid. The fluid pushed out from the liquid chamber 41 is ejected from the nozzle 4 at the distal end of the ejection tube 3.

FIG. 4 is a graph illustrating a transition of a capacity of a liquid chamber. The vertical axis indicates the capacity of the liquid chamber 41, and the capacity on an upper side in the drawing is larger than that on a lower side. The lateral axis indicates a transition of time and the time proceeds from a left side to a right side in the drawing. A first transitional line 50 indicates a transition in capacity when the capacity of the liquid chamber 41 is significantly changed. A second transitional line 51 indicates a transition in capacity when the capacity of the liquid chamber 41 is little changed.

The first transitional line 50 and the second transitional line 51 are repeated at the same cycle 52. The first transitional line 50 and the second transitional line 51 have a similar shape, and hence the transition of a change in capacity is described by the first transitional line 50. One cycle 52 is divided into a rising segment 53, a falling segment 54, and a pause segment 55. In the rising segment 53, the first transitional line 50 has a shape similar to a sine waveform. At this time, the voltage is applied to the piezoelectric element 47, and the piezoelectric element 47 is expanded. Accordingly, the diaphragm 44 moves in the first direction 48, and the capacity of the liquid chamber 41 is reduced. Then, the first liquid 12 of the liquid chamber 41 is moved to the outlet channel 42.

In the falling segment 54, the first transitional line 50 has a shape similar to a sine waveform. At this time, the voltage applied to the piezoelectric element 47 is decreased, and the piezoelectric element 47 is contracted. Accordingly, the diaphragm 44 moves in the second direction 49, and the capacity of the liquid chamber 41 is increased. Then, the first liquid 12 flows from the inlet channel 38 into the liquid chamber 41. The falling segment 54 is longer than the rising segment 53. Accordingly, the first liquid 12 flashes out to the outlet channel 42 and enters from the inlet channel 38 at a low speed. The pause segment 55 is a segment in which the piezoelectric element 47 maintains the contracted state. The cycle 52 may be adjusted by changing the length of the pause segment 55.

An amount of a change in capacity of the first transitional line 50 is defined as a first amount of change 50a, and an amount of a change in capacity of the second transitional line 51 is defined as a second amount of change 51a. The amounts of the changes of the first amount of change 50a and the second amount of change 51a can be adjusted by the control device 13 controlling the piezoelectric element 47.

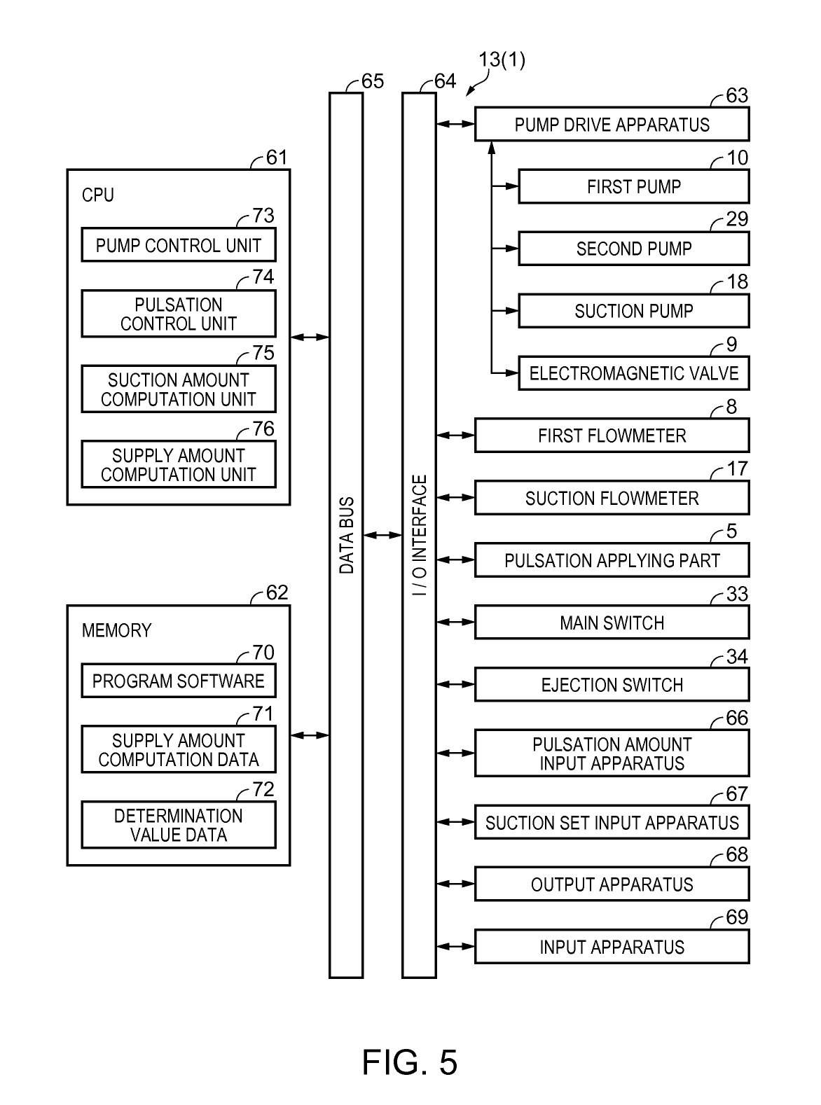

FIG. 5 is an electric control block diagram of the liquid ejection device 1. In FIG. 5, the liquid ejection device 1 is provided with the control device 13 configured to control the operation of the liquid ejection device 1. The control device 13 is provided with a CPU 61 (central processing unit) configured to perform various types of arithmetic treatments as a processor, and a memory 62 configured to store various types of information. A pump drive apparatus 63, the first flowmeter 8, the suction flowmeter 17, and the pulsation applying part 5 are connected to the CPU 61 via an I/O interface 64 and a data bus 65. In addition, the main switch 33, the ejection switch 34, a pulsation amount input apparatus 66, a suction setting input apparatus 67, an output apparatus 68, and an input apparatus 69 are connected to the CPU 61 via the I/O interface 64 and the data bus 65.

The pump drive apparatus 63 is configured to drive the first pump 10, the second pump 29, the suction pump 18, and the electromagnetic valve 9. The pump drive apparatus 63 inputs an instruction signal of the CPU 61. Then, the pump drive apparatus 63 drives the first pump 10, the second pump 29, and the suction pump 18 at a pressure or a flow rate indicated by the instruction signal. In addition, the pump drive apparatus 63 is configured to drive the electromagnetic valve 9 to open and close the valve.

The pulsation amount input apparatus 66 is configured to allow the operator to input an amount of variation in pulsation of the first liquid 12. The pulsation amount input apparatus 66 is configured to set an amount of the change in capacity of the liquid chamber 41 to the first amount of change 50a and the second amount of change 51a (FIG. 4), for example. The pulsation amount input apparatus 66 may be composed of a variable resistor, a circuit configured to convert a value of resistance of the variable resistor into a voltage, and a plurality of switches, for example.

The suction setting input apparatus 67 is configured to allow the operator to set a suction amount of the liquid sucked from the suction port 15. The output apparatus 68 includes a light and a speaker which notify abnormalities, and an apparatus configured to communicate with an external computer by cable or by radio, in addition to a liquid-crystal display. Accordingly, the control device 13 is capable of outputting the state of the liquid ejection device 1 and the setting state set by the operator.

The input apparatus 69 includes an apparatus configured to communicate with the external computer by cable or by radio in addition to a keyboard, a mouse-type input apparatus, and a pen-type input apparatus. Various types of data is input into the memory 62 by these input apparatuses 69.

The memory 62 is a concept including a semiconductor memory such as a RAM and a ROM, and an external storage device such as a hard disk and a DVD-ROM. Functionally, a storage area configured to store program software 70 in which a control procedure of the operation of the liquid ejection device 1 is described and a storage area configured to store supply amount computation data 71, which is to be used when computing a supply amount of the second liquid 31 are set. In addition, a storage area for storing determination value data 72, which is data used for determination when performing various types of control, is set. In addition, a storage area configured to function as a work area or a temporary file for the CPU 61 or other various storage areas are set.

The CPU 61 is configured to control ejection of the first liquid 12 from the nozzle 4 of the handpiece 2 in accordance with the program software 70 stored in the memory 62. As a specific function realizing unit, a pump control unit 73 is provided. The pump control unit 73 performs control to output an instruction signal to the pump drive apparatus 63, and drive the first pump 10, the second pump 29, and the suction pump 18 to cause the first liquid 12 and the second liquid 31 to flow and to be sucked. In addition, the pump control unit 73 controls the first liquid 12 and the second liquid 31 to stop the flow and to start the flow by opening and closing the electromagnetic valve 9.

In addition, the CPU 61 includes a pulsation control unit 74. The pulsation control unit 74 inputs an amount of pulsation set by the pulsation amount input apparatus 66. Then, the pulsation control unit 74 controls the amount of the variation of the liquid chamber 41 by controlling the piezoelectric element 47 of the pulsation applying part 5.

In addition, the CPU 61 includes a suction amount computation unit 75 as an adjusting unit. The suction amount computation unit 75 computes an adequate suction amount in accordance with the supply amount of the second liquid 31. The suction amount computation unit 75 computes a total flow rate of the liquid, which is a sum of a flow rate of the first liquid 12 ejected from the nozzle 4 and a flow rate of the second liquid 31 supplied from the supply port 24. The suction amount computation unit 75 then computes the suction amount, which is a flow rate of a sucked liquid so that the flow rate of the liquid sucked from the suction port 15 becomes smaller than the total flow rate of the liquid. Then, the computed suction amount is output to the pump control unit 73.

In addition, the CPU 61 includes a supply amount computation unit 76 as the adjusting unit. The supply amount computation unit 76 computes the supply amount of the second liquid 31 supplied from the supply port 24 by using a capacity supply amount correlation line 56 (FIG. 4B) on the basis of the amount of change of the capacity of the liquid chamber 41. Then, the supply amount is output to the pump control unit 73. The supply amount is controlled by the supply amount computation unit 76 and the pump control unit 73.

In the first embodiment, the respective functions described above are realized by the program software by using the CPU 61. However, in the case where the above-described respective functions may be realized by a single electronic circuit (hardware) which does not employ the CPU 61, such an electronic circuit may be used.

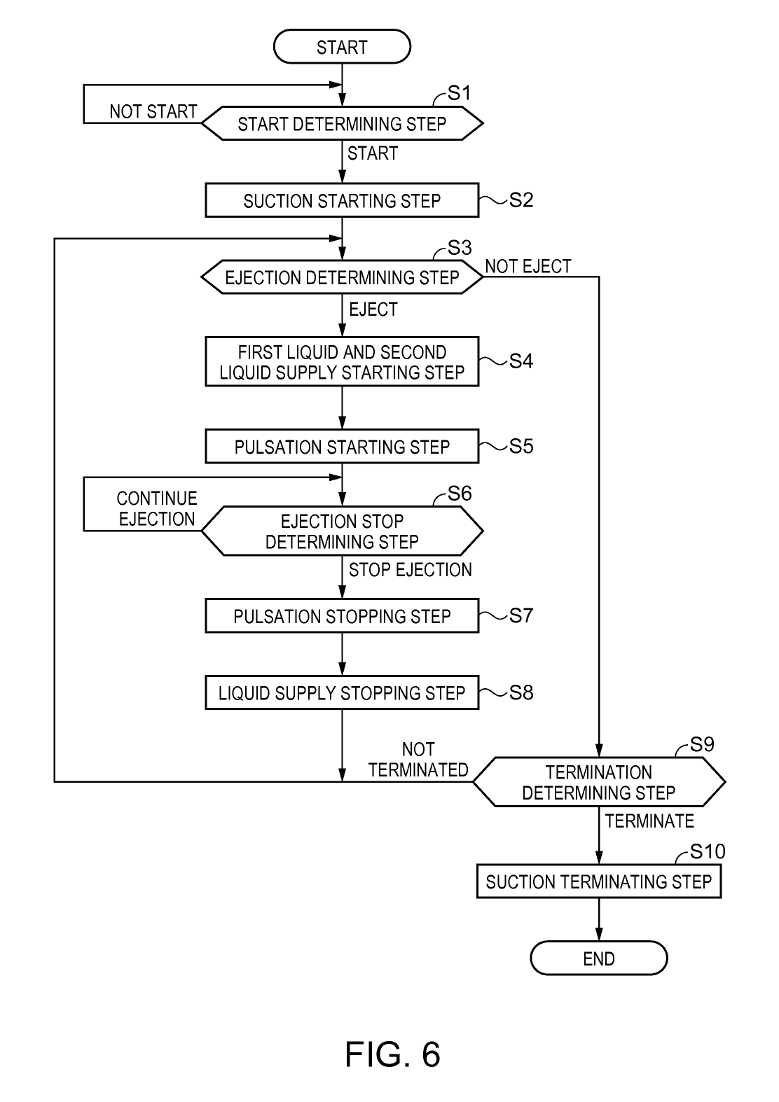

Subsequently, a method of cutting a surface of the living body (not illustrated) by using the above-described liquid ejection device 1 will be described with reference to FIG. 6. FIG. 6 is a flowchart of the cutting method that cuts the surface of the living boy.

In the flowchart illustrated in FIG. 6, Step S1 corresponds to a start determination process. In this step, whether the operator turns the main switch 33 ON is determined. The procedure waits until the operator turns the main switch 33 ON, and when the operator turns the main switch 33 ON, the procedure then goes to Step S2.

Step S2 corresponds to a suction starting step. In this step, the pump control unit 73 causes the pump drive apparatus 63 to drive the suction pump 18. At this time point, the second liquid 31 is not supplied, and hence the suction pump 18 sucks air. Subsequently, the procedure goes to Step S3.

Step S3 corresponds to an ejection determining step. In this step, the CPU 61 detects whether the ejection switch 34 is ON or OFF. When the ejection switch 34 is turned ON, the CPU 61 determines to eject the first liquid 12, and then the procedure goes to Step S4. When the ejection switch 34 is OFF, the CPU 61 determines not to eject the first liquid 12, and then the procedure goes to Step S9.

Step S4 corresponds to a first liquid and second liquid supply starting step. In this step, the pump control unit 73 causes the pump drive apparatus 63 to drive the first pump 10 and the second pump 29. Then, the first pump 10 flows the first liquid 12 toward the pulsation applying part 5. The second pump 29 causes the second liquid 31 to flow to the second tube 25. Subsequently, the procedure goes to Step S5.

Step S5 corresponds to a pulsation starting step. In this step, the pulsation control unit 74 inputs a set amount of pulsation set by the pulsation amount input apparatus 66. The amount of pulsation is set by the operator in advance. The operator may change the amount of pulsation in the course of the operation. The pulsation control unit 74 drives the pulsation applying part 5 so that the first liquid 12 is ejected at a set amount of pulsation. Subsequently, the procedure goes to Step S6.

Step S6 corresponds to an ejection stop determining step. In this step, the CPU 61 detects whether the ejection switch 34 is ON or OFF. When the ejection switch 34 is turned ON, the CPU 61 determines to continue ejection of the first liquid 12, and then the procedure goes to Step S7. When the ejection switch 34 is turned OFF, the CPU 61 determines to stop ejection of the first liquid 12, and then the procedure goes to Step S7.

Step S7 corresponds to a pulsation stopping step. In this step, the pulsation control unit 74 stops the driving of the pulsation applying part 5. Subsequently, the procedure goes to Step S8.

Step S8 corresponds to a liquid supply stopping step. In this step, the pump control unit 73 causes the pump drive apparatus 63 to stop driving of the first pump 10 and the second pump 29. Accordingly, ejection of the first liquid 12 from the nozzle 4 is stopped, and a supply of the second liquid 31 from the supply port 24 is also stopped. Subsequently, the procedure goes to Step S3.

Step S6 to Step S8 are performed substantially simultaneously. In the case of stopping the ejection of the first liquid 12, the pump control unit 73 controls the first pump 10 and the second pump 29 so that the second liquid 31 is not supplied from the supply port 24. Therefore, the second liquid 31 is not supplied wastefully at the time of non-ejection of the first liquid 12. Therefore, the amount of consumption of the second liquid 31 may be reduced.

Step S9 corresponds to a termination determining step. In this step, whether the operator turns the main switch 33 OFF is determined. When the operator keeps the main switch 33 ON, it is determined that the operation is continued and not to be terminated, and the procedure goes to Step S3. When the operator turns the main switch 33 OFF, the operation is determined to be terminated. Subsequently, the procedure goes to Step S10.

Step S10 corresponds to a suction terminating step. In this step, the pump control unit 73 causes the pump drive apparatus 63 to stop the driving of the suction pump 18. With the steps as described thus far, the process of cutting the surface of the living body is terminated.

As described above, according to the liquid ejection device 1 of the first embodiment, with the configuration in which the second liquid 31 is supplied additionally into the suction tube 16, the excised living tissue or the like may be effectively flowed (sucked) with a sufficient water amount (water flow) containing the second liquid 31 supplied additionally. Specifically, the second tube 25 is connected to the supply port 24 formed on the suction tube 16 to cause the second liquid 31 for adding the amount of washing water from the second tube 25 to flow into the suction tube 16. Accordingly, the excised tissue or the like may be restricted from being accumulated in the suction tube 16, and hence clogging of the suction tube 16 may be prevented.

Specifically, even with the liquid ejection device 1 configured to eject a liquid in a pulsed manner which is subjected to a decrease of the amount of washing water in comparison with the continuous-ejection-type liquid ejection device configured to eject the first liquid 12 such as the normal saline solution continuously, the suction tube 16 may reliably be prevented from being clogged.

### Second Embodiment

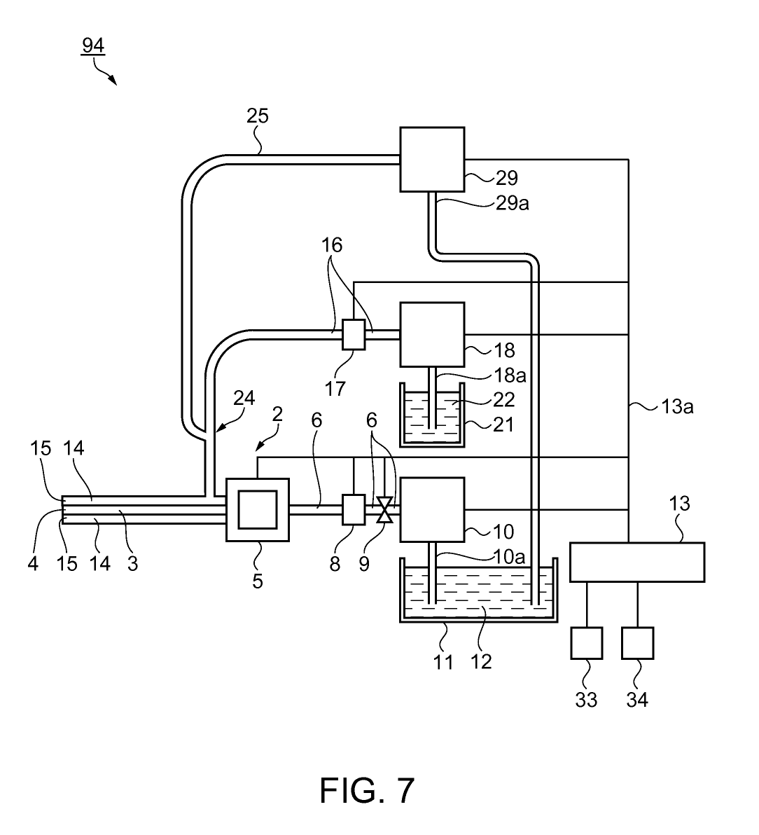

Subsequently, another embodiment of the liquid ejection device will be described with reference to FIG. 7. FIG. 7 is a block diagram illustrating a configuration of the liquid ejection device. The second embodiment is different from the first embodiment in that the first pump 10 and the second pump 29 illustrated in FIG. 1A cause the same liquid to flow. Description of the same points as the first embodiment will be omitted.

In other words, as illustrated in FIG. 7, a liquid ejection device 94 includes the first liquid 12 stored in the first water storage tank 11 in the second embodiment. The water inlet tube 10a of the first pump 10 and the water inlet tube 29a of the second pump 29 are connected to the first water storage tank 11. Therefore, the first pump 10 and the second pump 29 cause the first liquid 12 to flow. The first liquid 12 is ejected from the nozzle 4, and the first liquid 12 is supplied also from the supply port 24.

As described above, the second embodiment has the following advantageous effects in addition to the effect of the embodiment described above.

(1) According to the second embodiment, the first liquid 12 and the second liquid 31 do not have to be prepared separately. Therefore, a container for the second liquid 31 is not necessary, and hence the liquid ejection device 94 may be configured to be a compact apparatus.

### Modification of Second Embodiment

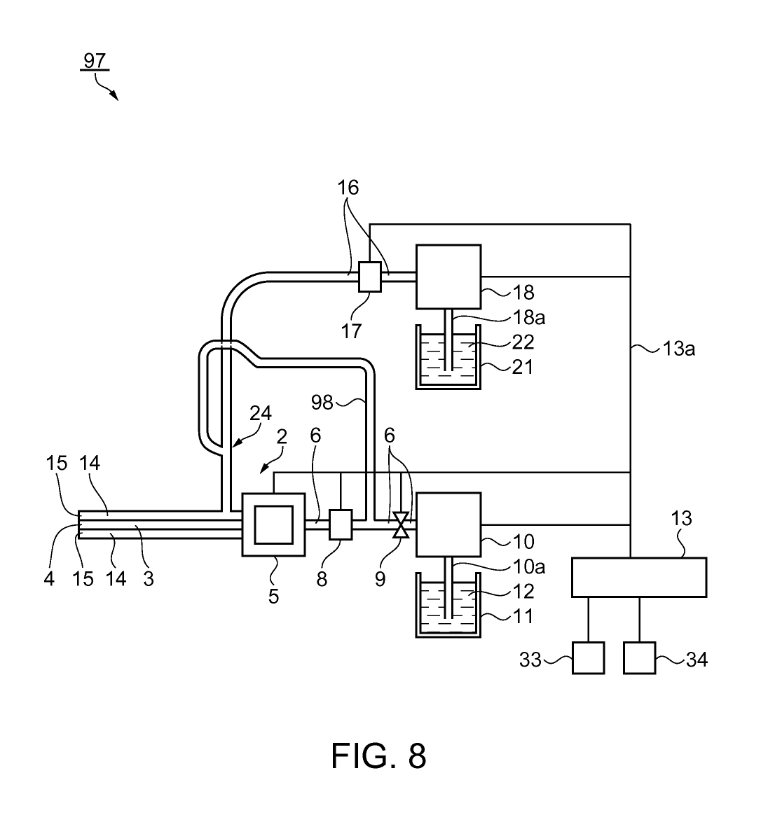

Subsequently, a modification of the liquid ejection device will be described with reference to FIG. 8. FIG. 8 is a block diagram illustrating a configuration of the liquid ejection device. This modification is different from the first embodiment in that the first pump 10 illustrated in FIG. 1A causes the same liquid to flow in the ejection tube 3 and the second tube 25. Description of the same points as the first embodiment will be omitted.

In other words, in this modification, a liquid ejection device 97 includes a third tube 98 connected to the first tube 6 which connects the first pump 10 and the pulsation applying part 5 as illustrated in FIG. 8. Specifically, the third tube 98 is connected to the first tube 6 between the first flowmeter 8 and the electromagnetic valve 9. The third tube 98 connects the first tube 6 and the suction tube 16.

The first liquid 12 flowed by the first pump 10 is supplied to the pulsation applying part 5 and the third tube 98. The first pump 10 causes the first liquid 12 to flow. The first liquid 12 is ejected from the nozzle 4, and the first liquid 12 is supplied also from the supply port 24 into the suction tube 16.

As described above, this modification has the following advantageous effects in addition to the effect of the embodiment described above.

(1) According to this modification, the first liquid 12 and the second liquid 31 do not have to be prepared separately. Therefore, the container for the second liquid 31 is not necessary, and hence the liquid ejection device 97 may be configured to be a compact apparatus.

(2) According to this modification, the liquid ejection device 97 causes the first pump 10 to flow the first liquid 12 to the pulsation applying part 5 and the third tube 98. Therefore, in comparison with the configuration having a pump which causes the third tube 98 to flow the first liquid 12 in addition to the first pump 10 which causes the pulsation applying part 5 to flow the first liquid 12, the number of the pumps may be reduced. Therefore, the liquid ejection device 97 may be formed into a compact apparatus.

### Third Embodiment

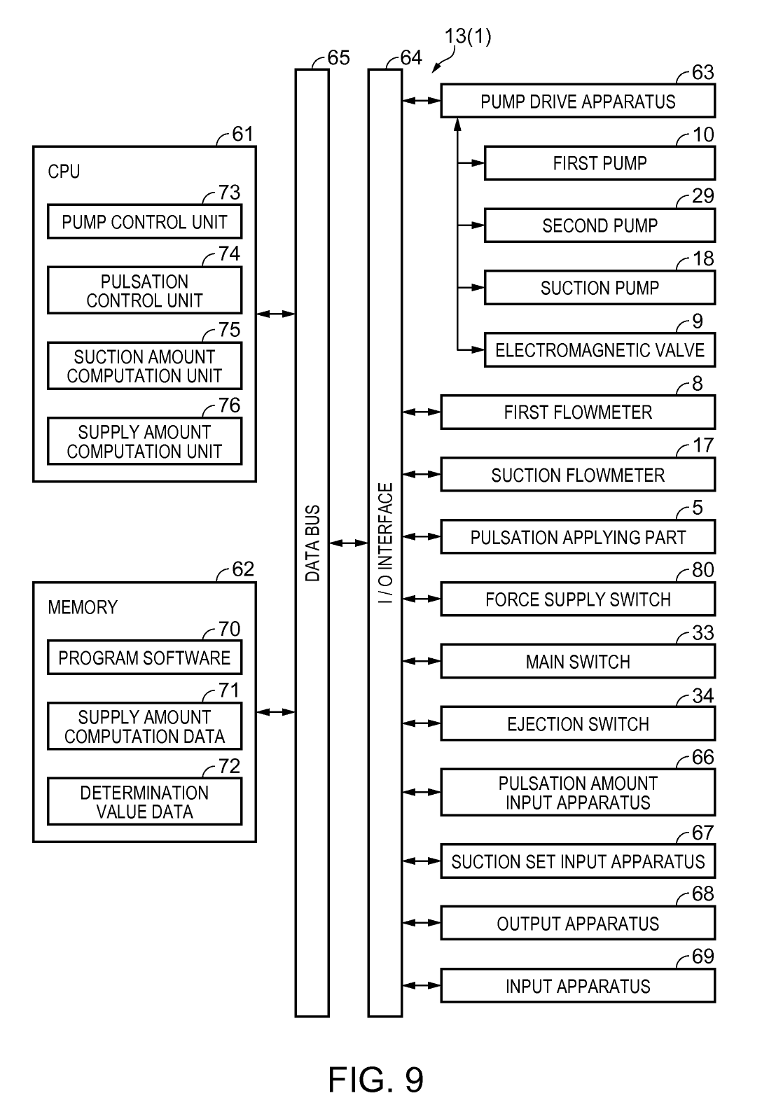

Subsequently, the liquid ejection device 1 will be described in conjunction with a third embodiment of the control device 13 as the controller with reference to FIG. 9. FIG. 9 illustrates a schematic configuration drawing of the control device 13. Description of the same points as the first embodiment will be omitted. The control device 13 is provided with a force supply switch 80, and is configured to be capable of drive-controlling the second pump 29 and the suction pump 18 by operating the force supply switch 80. Therefore, even when the first liquid 12 is not ejected, the second liquid 31 is supplied from the supply port 24 into the suction tube 16 via the second tube 25, and the interior of the suction tube 16 may be sucked.

By operating the force supply switch 80, even when the first liquid 12 is not ejected, the living tissue or the like remaining in the suction tube may be sucked by supplying the second liquid 31 to the second tube 25 and the suction tube 16 and sucking the same, and clogging of the suction tube may be restrained.

The third embodiment is not limited to the embodiments described above, and various changes or improvements may be applied by a person having a normal knowledge in the corresponding field within the technical scope of the invention. For example, the above-described embodiments and the modification may be applied to the liquid ejection device of the continuously ejecting type which ejects the first liquid 12 such as the normal saline solution continuously. The larger the amount of the washing water, the more reliably and efficiently the excised living tissue or the like may be flushed away (sucked out).

What is claimed is:

1. A liquid ejection device comprising:
a liquid ejection opening configured to eject a first liquid;
a liquid suction opening arranged in the vicinity of the liquid ejection opening; and
a suction unit configured to suck liquid from the liquid suction opening via a suction tube,
wherein the liquid ejection device includes a second liquid supply unit, and a second liquid can be supplied into the suction tube.

2. The liquid ejection device according to claim 1, further comprising:
a switch configured to switch between ejection and non-ejection of the first liquid; and
a controller configured to control to make the second liquid supply unit start a supply of the second liquid when starting ejection of the first liquid, and to stop the supply of the second liquid from the second liquid supply unit when stopping ejection of the first liquid.

3. The liquid ejection device according to claim 1,
wherein the first liquid is identical with the second liquid.

4. The liquid ejection device according to claim 1,
wherein the second liquid includes an enzyme which decomposes a protein or a lipid.

5. The liquid ejection device according to claim 1,
wherein a supply flow rate of the second liquid supplied from the second liquid supply unit is 5 milliliters or more per minute.

6. The liquid ejection device according to claim 1,
wherein the liquid ejection device further includes a force supply switch for allowing the second liquid to be supplied from the second liquid supply unit when the liquid is not ejected.
